(19) United States
(12) Patent Application Publication
(10) Pub. No.: US 2013/0045287 A1
(43) Pub. Date: Feb. 21, 2013

(54) Treatment for Concussion using Gallium Compounds

(71) Applicant: Jan M. Troup, The Woodlands, TX (US)

(72) Inventor: Jan M. Troup, The Woodlands, TX (US)

(21) Appl. No.: 13/584,407

(22) Filed: Aug. 13, 2012

Related U.S. Application Data
(60) Provisional application No. 61/525,660, filed on Aug. 19, 2011.

(51) Int. Cl. A61K 33/24; A61P 25/00; A61K 31/28

(52) U.S. Cl. 424/650; 514/492

(57) ABSTRACT

Disclosed is a method of treating concussion or other brain trauma in humans or other mammals by administering an effective dose of a gallium compound or gallium complex to the injured human or other mammal. The gallium compound may comprise, e.g., gallium nitrate, a docosahexaenonic acid salt of gallium with one to three of the gallium ligands being from docosahexaenonic acid, or other gallium compound or complex or combinations thereof. The gallium compound or complex may be administered orally, by spray, intravenously, subcutaneously, transdermally, intramuscularly, mucoadhesively or through oral or nasal inhalation. A transdermal patch containing the gallium compound may be applied after onset of the concussion or other brain trauma to permit rapid delivery of the compound through the skin. The gallium compound is preferably administered in an amount sufficient to maintain steady state blood concentrations. A preferred dosing is about 0.5 to about 20.0 mg/kg/day of body weight.

### CROSS-REFERENCE TO RELATED APPLICATIONS

This application claims the benefit of the filing date of and priority to U.S. Provisional Application Ser. No. 61/525,660 entitled “Treatment for Concussion using Gallium Compounds” and filed Aug. 19, 2011, Confirmation No. 9547. Said application is incorporated by reference herein.

### STATEMENT REGARDING FEDERALLY SPONSORED RESEARCH OR DEVELOPMENT

Not Applicable.

### BACKGROUND OF THE INVENTION

### Field of the Invention

Traumatic brain injury is the leading cause of death in people under the age of 45 in the United States. Data from The Center for Disease Control (CDC) indicate that approximately every 22 seconds someone in the United States sustains a serious traumatic brain injury. There are about 3.8 million sports and recreational related concussions in the United States every year with 1,365,000 emergency room visits and 52,000 deaths.

Giza, C. C. and Hovda, D. A. The Neurometabolic Cascade of Concussion. J. Athletic Training. 2001, 36(3), 228-235, describe the series of events following a concussion, including the associated chemical pathways. The trauma of cerebral concussion generates compressive, tensile, and rotational forces resulting in diffuse axonal injury. Immediately following the injury there is a sudden intracellular efflux of potassium and an influx of calcium ions producing a hypercalcemia condition in the brain. The concussed brain goes into a period of depressed metabolism with continued increases in calcium potentially impairing mitochondrial oxidative metabolism. The calcium accumulation can lead to cell death and disrupt neurofilaments and microtubules. The calcium accumulation is seen within hours of a concussion and persists for two to four days after an event. Additionally, cerebral swelling as a result of calcium and sodium influx occurs post concussion and further exposes the patient to additional risk.

Concussion treatment usually consists of only patient rest for days to months to allow for the brain to heal. Docosahexaenoic acid (DHA) has been found to both successfully treat the brain damage of patients after concussion but also to offer protection and prevent neuron damage if taken prior to a concussion in rat studies. See, for example, Bailes, J. E. and Mills, J. D. Docosahexaenoic acid reduces traumatic brain injury in a rodent head injury model. J. Neurotrauma. 2010, September, 27, 1617-1624; Mills, J. D.; Bailes, J. E.; Sedney, C. L.; Hutchins, H. and Sears, B. Omega-3 fatty acid supplementation and reduction of traumatic axonal injury in a rodent head injury model. J. Neurosurg. 2011, January, 114(1), 77-84 and Bailes, U.S. Pat. App. Pub. No. US2011/0086914, Apr. 14, 2011, incorporated herein by reference.

To date no proven pharmaceutical treatment has been found that stops or reduces the chemical imbalances that result from a concussion to limit the brain damage, and therefore, the present invention is focused on providing such desired treatment.

According to National Institute of Neurological Disorders and Stroke (NINDS), “Discussion on Traumatic Brain Injury: Hope through Research”, Apr. 15, 2011, “[o]ne area of research that shows promise is the study of the role of calcium ion influx into the damaged neuron as a cause of cell death and general brain tissue swelling. Calcium enters nerve cells through damaged channels in the axon's membrane. The excess calcium inside the cell causes the axon to swell and also activates chemicals, called proteases, that break down proteins. One family of proteases, the calpains, are especially damaging to nerve cells because they break down proteins that maintain the structure of the axon. Excess calcium within the cell is also destructive to the cell's mitochondria, structures that produce the cell's energy. Mitochondria soak up excess calcium until they swell and stop functioning. If enough mitochondria are damaged, the nerve cell degenerates. Calcium influx has other damaging effects: it activates destructive enzymes, such as caspases that damage the DNA in the cell and trigger programmed cell death, and it damages sodium channels in the cell membrane, allowing sodium ions to flood the cell as well. Sodium influx exacerbates swelling of the cell body and axon. NINDS researchers have shown, in both cell and animal studies that giving specialized chemicals can reduce cell death caused by calcium ion influx.” NIH. www.ninds.nih.gov/disorders/tbi/detail_tbi.htm.

A recent approach in the treatment of traumatic brain injury is research using neuropathic pain blockers known as N-type voltage-gated calcium channel blockers (N-type VGCC blockers). N-type VGCC blockers such as SNX-111 (Zirconotide) (Samii et al.) and SNX-185 (Lee et al.; Shahlaie et al.) have been studied in rats and found to be effective as neuropathic pain blockers and as neuroprotective agents in traumatic brain injury. See, Samii, A.; Badie, H.; Fu, K.; Luther, R. R. and Hovda, D. A. Effects of an N-type calcium channel antagonist (SNX 111; Ziconotide) on calcium-45 accumulation following fluid-percussion injury. J. Neurotrauma. 1999, October, 16(10), 879-92; Lee, L. L.; Galo, E.; Lyeth, B. G.; Muizelaar, J. P. and Berman, R. F. Neuroprotection in the rat lateral fluid percussion model of traumatic brain injury by SNX-185, an N-type voltage-gated calcium channel blocker. Exp. Neurol. 2004, November, 190(1), 70-8; and Shahlaie, K.; Lyeth, B. G.; Gurkoff, G. G.; Muizelaar, J. P. and Berman, R. F. Neuroprotective Effects of Selective N-Type VGCC Blockade on Stretch-Injury-Induced Calcium Dynamics in Cortical Neurons. J. Neurotrauma. 2010, January, 27(1), 175-187.

Additionally, SNX-111 was recently found to be biologically active as a therapeutic agent in humans for the treatment of neuropathic pain and for neuroprotection after ischemic brain injury. McGuire, D.; Bowersox, S.; Fellmann, J. D. and Luther, R. R. Sympatholysis After Neuron-Specific, N-Type, Voltage-Sensitive Calcium Channel Blockade: First Demonstration of N-Channel Function in Humans. J. Cardiovascular Pharmacology. 1997, September, 30(3), 400-403.

The rat studies showed a reduction of trauma-induced calcium accumulation in the cerebral cortex and especially the hippocampus tissue. This work using N-type VGCC neuropathic pain blockers is encouraging for treating posttraumatic calcium accumulation using other neuropathic pain blockers that limit the calcium levels of traumatic brain injury.

Calcitonin and gallium compounds, are effective drugs for reducing calcium levels in hypercalcemia patients and both have been shown also to be effective in treating neuropathic pain like N-type VGCC blockers. Calcitonin was studied for spinal cord injury (Humble, S. R. Calcitonin for acute neuropathic pain associated with spinal cord injury. Anaesth. Intensive Care. 2011, July, 39(4), 682-86) and gallium compounds have been studied for a variety of neurological aliments (Bernstein, U.S. Pat. No. 8,168,214). A number of theories exist as to the mechanism of activity in the case of calcitonin, involving the serotonegic system (Humble, S. R., supra) or normalization of the sodium channel expression (Ito, A.; Takeda, M.; Yoshimura, T.; Komatsu, T.; Ohno, T.; Kuriyama, H.; Matsuda, A. and Yoshimura, M. Anti-hyperalgesic effects of calcitonin on neuropathic pain interacting with its peripheral receptors. Mol. Pain. 2012, Jun. 7; 8(1):42 [Epub ahead of print]) but much less is known regarding the mechanism for gallium compounds. Calcitonin and gallium compounds each have similarity in activity with the known neuroprotection of N-type VGCC blockers making them promising for the treatment of traumatic brain injury.

Gallium compounds are known to both rapidly reduce edema in animals and humans. For example Gerber et al., U.S. Pat. No. 5,700,487, incorporated herein by reference, discloses a method of treating pulmonary inflammation in mammals, comprising administering an effective amount of a pharmaceutically acceptable gallium compound and wherein said gallium is elected from the group consisting of gallium nitrate, gallium citrate, gallium chloride, gallium carbonate, gallium acetate, gallium tartrate, gallium oxalate, gallium oxide, gallium arsenide and hydrated gallium oxide.

Gallium compounds are used to inhibit bone calcium loss (hypercalcemia) in cancer patients. See Warrell, R. P.; Brockman, R. S.; Coonley, C. J.; Isaacs, M. and Staszewski, H. Gallium nitrate inhibits calcium resorption from bone and is effective treatment for cancer-related hypercalcemia. J. Clinical Investigation. 1984, 73, 1487-1490; and Todd, P. A. and Fitton, A. Gallium nitrate. A review of its pharmacological properties and therapeutic potential in cancer related hypercalcemia. Drugs. 1991, August, 42(2), 261-73 regarding use of gallium nitrate.

Also, Bradley, et al., U.S. Pat. No. 5,196,412, incorporated herein by reference, describes compounds of gallium (III) which can be given orally to achieve high serum levels of gallium (III) for the treatment of hypercalcemia of malignancy and related disorders of bone metabolism. Complexes of gallium (III) of the formula (I)

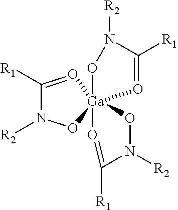

wherein R₁ is C₁-C₈ n-alkyl and R₂ is H or C₁-C₂ alkyl, or R₁ and R₂ together form tetra- or penta-methylene and wherein said complex is useful in increasing calcium content of bone tissue and decreasing bone resorption, are used in an amount sufficient to cause an increase in calcium content of said bone and to cause decreased bone resorption.

In accordance with the present invention, these gallium compounds may be the ideal treatment for concussion.

Julian, U.S. Pat. No. 7,354,952, incorporated herein by reference, discloses novel pharmaceutical gallium compositions, including gallium complexes having increased oral bioavailability relative to uncomplexed gallium salts. Such compositions are useful in the treatment of conditions and diseases in which inhibition of abnormally increased calcium resorption is desired, including cancer, hypercalcemia, osteoporosis, osteopenia and Paget's disease.

Jiang et al., U.S. Pat. No. 7,119,217, incorporated herein by reference, discloses novel tri(alkylcarboxylato) gallium (III) compounds, exemplified by tripalmitato gallium (III), methods for making them, pharmaceutical compositions containing them, and methods of using the pharmaceutical compositions. These compounds may be useful especially since a DHA is a member of this family of compounds.

The use of gallium compounds for the treatment of traumatic brain injury (TBI) or concussion is not described in the scientific literature, medical literature or prior art. Gallium compounds and complexes have not been the subject of extensive medical research or treatment except for treating hypercalcemia in cancer patients.

In an early study by Bockman et al., U.S. Pat. No. 4,704,277, it was shown that doses of 100-300 mg/sq mm m/day of gallium nitrate over 5-7 days reduce calcium excretion by 70±18%. In particular, Bockman et al. reported that: Seventeen infusions were administered to fifteen patients, five of whom were hypercalcemic. Gallium nitrate was administered as a continuous infusion at daily doses ranging from 100-300 mg/sq mm m/d, over 5-7 days. Bone turnover was assessed by serial measurements of urinary levels of Ca²⁺, hydroxyproline (OHP), and creatinine (Cr), as well as the serum levels of osteocalcin (BGP). The urinary 24 hour Ca²⁺ excretion hours after administration of the drug, was markedly reduced after each infusion in all 15 patients. Mean reduction was 70±18%. Eight patients with increased bone turnover (i.e., a urinary OHP/Cr ratio greater than 6.0), received eleven drug infusions. All patients showed a decrease in OHP/Cr. Mean reduction was 49±22%. Six patients with bone turnover which was not elevated showed no significant change in this ratio. These data show that gallium treatment reduces accelerated bone loss in cancer patients with bone matastases. Cytotoxicity studies were undertaken to determine if gallium nitrate was cytotoxic to bone tissue. Histological studies show no evidence of cytotoxic effect at the light microscopic level. Additionally, stained sections show no alteration in matrix or mineral components. Little change in cell numbers or viability was seen when bone cell models were treated for 48 hours with up to 25 μM of gallium nitrate. At this dose and time, a 20% decrease in ³H-thymidine incorporation into DNA was seen. If gallium treated and untreated cells were labeled with ³H-amino acids, no differences were seen in the protein banding patterns on the SDS-PAGE gels of the cell homogenates. These results show that the action of gallium is not a consequence of a cytotoxic effect on bone cells.

In the cancer application, the gallium appears to inhibit calcium resorption from bone and the exact mechanism is unknown although it could be a reaction with the calcium apatite bone structure binding the calcium into a harder bone structure. An FDA-approved product called Ganite® (gallium nitrate injection) (from Genta, Inc., www.genta.com, Berkeley Heights, N.J.) is currently being used for these cancer treatment applications. According to Genta, Inc.'s website for the Ganite® product (www.ganite.com), Ganite® is indicated for the treatment of clearly symptomatic cancer-related hypercalcemia that has not responded to adequate hydration. In general, patients with a serum calcium concentration (corrected for albumin) <12 mg/dL would not be expected to be symptomatic. Mild or asymptomatic hypercalcemia may be treated with conservative measures (i.e., saline hydration, with or without diuretics). In the treatment of cancer-related hypercalcemia, it is important first to establish adequate hydration, preferably with IV saline, in order to increase the renal excretion of calcium and correct dehydration caused by hypercalcemia.

Ganite (Gallium nitrate injection) is a clear, colorless, odorless, sterile solution of gallium nitrate, a hydrated nitrate salt of the group IIIa element, gallium. Gallium nitrate is formed by the reaction of elemental gallium with nitric acid, followed by crystallization of the drug from the solution. The stable, nonahydrate, Ga(NO₃)₃.9H₂O is a white, slightly hygroscopic, crystalline powder of molecular weight 417.87, that is readily soluble in water. Each mL of Ganite (gallium nitrate injection) contains gallium nitrate 25 mg (on an anhydrous basis) and sodium citrate dihydrate 28.75 mg. The solution may contain sodium hydroxide or hydrochloric acid for pH adjustment to 6.0-7.0. (See website www.ganite.com/docs/30105901.BMR8.pdf).

The following efficacy study was reported by Genta, Inc. regarding its Ganite® product (www.ganite.com/hcp/efficacy_studies.shtml).

A randomized double-blind clinical study comparing Ganite® with calcitonin (for acute control of cancer-related hypercalcemia) was conducted in patients with a serum calcium concentration (corrected for albumin) ≧12.0 mg/dL following 2 days of hydration. Ganite was administered as a continuous intravenous infusion at a dose of 200 mg/m²/day for 5 days and calcitonin was administered intramuscularly at a dose of 8 I.U./kg every 6 hours for 5 days. Elevated serum calcium (corrected for albumin) was normalized in 75% (18 of 24) of the patients receiving Ganite and in 27% (7 of 26) of the patients receiving calcitonin (p=0.0016). The time-course of effect on serum calcium (corrected for albumin) is summarized in Table I.

TABLE I
(www.ganite.com/hcp/efficacy_studies.shtml)(last accessed
Aug. 17, 2011)
Change in Corrected Serum Calcium Concentration
by Time from Initiation of Treatment in a Randomized, Double-Blind
Study Comparing Ganite and Calcitonin

Mean Change in
Serum Calcium
Concentration
(mg/dL)*

| Time Period^{†} (hours) | Ganite | Calcitonin |
|---|---|---|
| 24 | −0.4 | −1.6^{‡} |
| 48 | −0.9 | −1.4 |
| 72 | −1.5 | −1.1 |
| 96 | −2.9^{‡} | −1.1 |
| 120 | −3.3^{‡} | −1.3 |

*Change from baseline in serum calcium concentration (corrected for albumin).
^{†}Time after initiation of therapy in hours.
^{‡}Comparison between treatment groups (p < 0.01).

The median duration of normocalcemia/hypocalcemia was 7.5 days for patients treated with Ganite and 1 day for patients treated with calcitonin. A total of 92% of patients treated with Ganite had a decrease in serum calcium (corrected for albumin) ≧2.0 mg/dL as compared to 54% of the patients treated with calcitonin (p=0.004).

An open-label, non-randomized study was conducted to examine a range of doses and dosing schedules of Ganite for control of cancer-related hypercalcemia. The principal dosing regimens were 100 and 200 mg/m²/day, administered as continuous intravenous infusions for 5 days. Ganite, at a dose of 200 mg/m²/day for 5 days was found to normalize elevated serum calcium levels (corrected for albumin) in 83% of patients as compared to 50% of patients receiving a dose of 100 mg/m²/day for 5 days. A decrease in serum calcium (corrected for albumin) ≧2.0 mg/dL was observed in 83% and 94% of patients treated with Ganite at dosages of 100 and 200 mg/m²/day for 5 days, respectively. There were no significant differences in the proportion of patients responding to Ganite when considering either the presence or absence of bone metastasis, or whether the tumor histology was epidermoid or nonepidermoid. (www.ganite.com/docs/30105901.BMR8.pdf).

From this Ganite product study it appears that an effect dose of gallium nitrate to normalize elevated serum calcium levels by 2.0 mg/dL was achieved with 200 mg/m²/day.

Gallium nitrate is also described to inhibit various forms of arthritic inflammation as taught by Matkovic et al., U.S. Pat. No. 5,175,006 and other forms of inflammation such as pulmonary inflammation taught by Gerber et al., U.S. Pat. No. 5,700,487, in humans and animals. Gallium compounds are also powerful antibacterial and anti-pathogenic agents as taught by Schlesinger et al., U.S. Pat. No. 6,203,822, which further discloses the use of gallium-containing compounds to inhibit intracellular pathogens including pathogens that are members of the genus Mycobacteria, Legionella, Histoplasma, and Leishmania and to organisms causing chronic pulmonary infection such P. aeruginosa. Additionally, gallium nitrate has been shown to be an effective for wound treatment as taught by Rogosnitzky, U.S. Pat. App. Pub. No. 20110104246, as a pharmaceutical composition and method for topical wound treatment by topical treatment with gallium salts, preferably gallium nitrate.

### BRIEF SUMMARY OF INVENTION

Gallium compounds are FDA approved to treat hypercalcemia in cancer patients and have also been shown to reduce pulmonary and arthritic and other forms of inflammation. Concussion or other traumatic brain injury results in an influx of calcium into the brain cells resulting in neuron damage and swelling. Gallium compounds have been shown to reduce serum calcium levels and reduce inflammation both of which are important following a brain injury. The brain damage process occurs rapidly after an injury and gallium compounds can be administered rapidly through a variety of methods immediately following an injury to reduce the tissue damage.

One embodiment of the present invention pertains to a method of treating concussion or other brain trauma in humans or other mammals, comprising administering an effective dose of a gallium compound or gallium complex (preferably pharmaceutically acceptable) to the injured human or other mammal. The gallium compound or complex may be selected from the group consisting of: gallium nitrate, gallium citrate, gallium chloride, gallium fluoride, gallium phosphate, gallium carbonate, gallium acetate, gallium tartrate, gallium oxalate, gallium formate, gallium oxide, gallium sulfate, gallium arsenide, gallium maltolate, gallium 8-quinolinolate and hydrated gallium oxide, gallium pyridinones, gallium succinate, gallium gluconate, gallium 3-hydroxy-4-pyrone, gallium palmitrate, the tridocosahexaenonic acid salt of gallium, or other tri(alkylcarboxylato) gallium (III) compounds, gallium prophyrins, gallium transferrins, gallium pyridoxal isonicotinoyl hydrazine, or combinations thereof. In a preferred embodiment, the gallium compound is gallium nitrate. In another embodiment, the gallium compound is a docosahexaenonic acid salt of gallium with one to three of the gallium ligands being from docosahexaenonic acid.

In practicing the methods disclosed herein, the gallium compound or complex may be administered orally, by spray, intravenously, subcutaneously, transdermally, intramuscularly, via bioadhesive or mucoadhesive patches (such as, e.g., a buccal patch) or through oral or nasal inhalation. In one embodiment, the gallium compound or complex is contained in a transdermal patch that may be applied after onset of the concussion or other brain trauma to permit rapid delivery of the compound through the skin. In one embodiment, the gallium compound or complex is administered after the onset of the concussion or brain trauma. The gallium compound or complex could be supplied from first aid kits for rapid treatment. These gallium compounds could be supplied to health professionals and emergency responders in a kit form for use in rapidly treating such concussions and traumatic brain injuries. Preferably, the first aid kits containing such gallium compounds would also be located at venues where such concussion and traumatic brain injuries are more likely to occur, i.e., sports venues, particularly those involving contact sports or the potential for head injuries, military combat operations, and EMT/fire response vehicles used in responding to vehicular-related accidents.

The gallium compound is preferably administered in an amount sufficient to maintain steady state blood concentrations. In one embodiment, the amount of gallium compound or complex administered is about 0.5 to about 20.0 mg/kg/day of body weight. In one embodiment, the amount of gallium nitrate administered is about 0.5 to about 20.0 mg/kg/day of body weight with the preferred amount being 200 mg/m²/day.

Although the methods described herein have applicability to the treatment of humans suffering concussions or other traumatic brain injuries, the methods may also be utilized on other mammals.

### DETAILED DESCRIPTION OF THE INVENTION

The focus of this invention is the treatment of concussion or other brain trauma with a pharmaceutically acceptable gallium compound. The action of the gallium is two-fold since the compounds are known to reduce hypercalcemia in cancer patients and also have been shown to reduce edema in animals and humans. The gallium compound can be one of a variety of such compounds that have been described in the literature with gallium nitrate being the most studied for hypercalcemia and tridocosahexaenoic acid salt of gallium (III) being of interest since DHA is effective in concussion treatment. The gallium compound can be administered orally, by spray, intravenously, subcutaneously, transdermally (such as, e.g., via passive transdermal single-layer and multi-layer drug-in-adhesive (DIA) technology offered by 3M, or, e.g., via a microneedle system such as the Microstructured Transdermal System (MTS) offered by 3M, buccal patches, and other known transdermal systems), intramuscularly or through oral or nasal inhalation. The gallium compound should be preferably administered immediately after the injury (or as soon thereafter as possible) so that its action can prevent or reduce damage from hypercalcemia or brain swelling. Orally, transdermally or nasal inhalation are preferred methods to apply treatment since they can be supplied from a kit at the time of the injury in a sufficient quantity to maintain a steady state blood concentration. The application of a transdermal patch on or near the injury would be especially effective since many gallium compounds are readily absorbed through the skin and the tissue response is rapid.

A gallium compound, with gallium nitrate being the compound of choice, is administered to a human or animal who has suffered a concussion or other brain trauma by the most convenient method, with oral, nasal inhalation or transdermally being the preferred methods, to reduce the immediate release of calcium in the brain. Due to the rapid onset of calcium influx post brain trauma or concussion, the timing of the gallium therapy is very important to stop tissue damage. The gallium compound should be administered as soon as a concussion or other brain trauma is determined to reduce or prevent hypercalcemia in the brain tissue. The influx of calcium starts within minutes of an injury and can last for 2-4 or more days.

The potential use of gallium compounds to reduce excess tissue calcium and prevent hypercalcemia in concussion or other brain trauma is the same approach as used in treating cancer patients for hypercalcemia. The additional benefit of rapid edema reduction makes concussion therapy with gallium compounds a good choice for sports related or military injury since it can be administered rapidly immediately following a concussion or brain trauma to reduce or eliminate the potential damage. Additionally, reducing calcium ion influx has been shown by NIH researchers to be successful in the treatment of traumatic brain injury in animals and humans. Gallium treatment does not appear to suppress the immune system like other anti-inflammatory agents such as steroids.

At sports events or on the battle field the gallium compound could be supplied from first aid kits for rapid treatment. Gallium nitrate and other gallium compounds are readily absorbed through the skin and have been utilized to treat or inhibit arthritis in animals. A rapid method of treating a brain injury would be to apply a transdermal patch from a first aid kit to an area of the head near the injury so that the treatment would reach the affected area as rapidly as possible. Additionally, the rapid anti-inflammatory action provided in minutes from the gallium compounds will reduce brain swelling and concurrent or future brain tissue damage that results from a concussion. In brain trauma, the gallium compound applied to an associated wound would also provide a powerful antibacterial effect to limit infection and stop or limit potential bleeding at the wound site.

The gallium compound with or without a citrate (such as in the Ganite formulation) should be administered in a sufficient amount, with 200 mg/m² or approximately 4 mg/kg of body weight for 1-7 days being the likely dose, to maintain steady state blood calcium concentrations for one or more days depending on the severity of the concussion. This is the standard FDA approved dose to reduce hypercalcemia in cancer patients. Brain tissue hypercalcemia can persists for up to one week.

Although gallium nitrate is the most common form of gallium for various treatments, other salts or complexes of gallium may be found to be absorbed more rapidly or may be more specific for reducing hypercalcemia and/or edema after brain trauma or concussion. One potential gallium compound in the family of tri(alkylcarboxylato) gallium (III) compounds is the docosahexaenonic acid (DHA) salt of gallium (III). This compound could be useful since DHA has been found to be a very effective treatment for concussion in rat studies and humans. The omega-3 content of the brain is 97% DHA and DHA appears to be essential to brain health, function and healing. The DHA salt of gallium might be an effective chemical conduit for gallium delivery since damaged brain tissue picks up DHA treatment rapidly to repair neuron damage. More research will be required to determine the best gallium salt or complex for brain trauma, and it is envisioned that a person of ordinary skill in the art, having the benefit of the present disclosure in this application, can derive optimal gallium compounds, doses and treatment delivery methods, these optimizations being considered to be part of the scope of the present invention.

All references referred to herein are incorporated herein by reference. While the apparatus, systems and methods of this invention have been described in terms of preferred or illustrative embodiments, it will be apparent to those of skill in the art that variations may be applied to the process and system described herein without departing from the concept and scope of the invention. All such similar substitutes and modifications apparent to those skilled in the art are deemed to be within the scope and concept of the invention. Those skilled in the art will recognize that the method and apparatus of the present invention has many applications, and that the present invention is not limited to the representative examples disclosed herein. Moreover, the scope of the present invention covers conventionally known variations and modifications to the system components described herein, as would be known by those skilled in the art.

### U.S. Patent References

| | | |
|---|---|---|
| 4,704,277 | Nov. 3, 1987 | Bockman et al. |
| 5,175,006 | Dec. 29, 1992 | Matkovic et al. |
| 5,196,412 | Mar. 23, 1993 | Bradley et al. |
| 5,700,487 | Dec. 23, 1997 | Gerber et al. |
| 6,203,822 | Mar. 20, 2001 | Schlesinger et al. |
| 7,119,217 | Oct. 10, 2006 | Jiang et al. |
| 7,354,952 | Apr. 8, 2008 | Julian |
| 8,168,214 | May 1, 2012 | Bernstein |

### U.S. Patent Application Publications

| | | |
|---|---|---|
| 2008/0113004 | May 15, 2008 | Bernstein |
| 20110086914 | Apr. 14, 2011 | Bailes |
| 20110104246 | May 5, 2011 | Rogosnitzky |

### Non-Patent Literature Documents

Bailes, J. E. and Mills, J. D. Docosahexaenoic acid reduces traumatic brain injury in a rodent head injury model. J. Neurotrauma. 2010, September, 27, 1617-1624.
Genta Inc., website excerpts regarding GANITE® gallium nitrate injection: www.ganite.com; www.ganite.com/docs/30105901.BMR8.pdf; http://www.ganite.com/hcp/efficacy_studies.shtml; www.genta.com/Products_and_Pipeline/Ganite/Ganite.html
Giza, C. C. and Hovda, D. A. The Neurometabolic Cascade of Concussion. J. Athletic Training. 2001, 36(3), 228-235.
Humble, S. R. Calcitonin for acute neuropathic pain associated with spinal cord injury. Anaesth. Intensive Care. 2011, July, 39(4), 682-86.
Ito, A.; Takeda, M.; Yoshimura, T.; Komatsu, T.; Ohno, T.; Kuriyama, H.; Matsuda, A. and Yoshimura, M. Anti-hyperalgesic effects of calcitonin on neuropathic pain interacting with its peripheral receptors. Mol. Pain. 2012, Jun. 7; 8(1):42 [Epub ahead of print].
Kaur, A. and Kaur, G. Mucoadhesive buccal patches based on interpolymer complexes of chitosan-pectin for delivery of carvedilol. Saudi Pharmaceutical Journal. 2012, 20, 21-27.
Lee, L. L.; Galo, E.; Lyeth, B. G.; Muizelaar, J. P. and Berman, R. F. Neuroprotection in the rat lateral fluid percussion model of traumatic brain injury by SNX-185, an N-type voltage-gated calcium channel blocker. Exp. Neurol. 2004, November, 190(1), 70-8.
McGuire, D.; Bowersox, S.; Fellmann, J. D. and Luther, R. R. Sympatholysis After Neuron-Specific, N-Type, Voltage-Sensitive Calcium Channel Blockade: First Demonstration of N-Channel Function in Humans. J. Cardiovascular Pharmacology. 1997, September, 30(3), 400-403.
Mills, J. D.; Bailes, J. E.; Sedney, C. L.; Hutchins, H. and Sears, B. Omega-3 fatty acid supplementation and reduction of traumatic axonal injury in a rodent head injury model. J. Neurosurg. 2011, January, 114(1), 77-84.
Nafee, N. A.; Boraie, N. A. and Ismail, F. A. Design and characterization of mucoadhesive buccal patches containing cetylpyridinium chloride. Acta Pharm. 2003, 53, 199-212.
NIH—National Institute of Neurological Disorders and Stroke (NINDS) Discussion on Traumatic Brain Injury: Hope through Research. Apr. 15, 2011. www.ninds.nih.gov/disorders/tbi/detail_tbi.htm
Samii, A.; Badie, H.; Fu, K.; Luther, R. R. and Hovda, D. A. Effects of an N-type calcium channel antagonist (SNX 111; Ziconotide) on calcium-45 accumulation following fluid-percussion injury. J. Neurotrauma. 1999, October, 16(10), 879-92.
Shahlaie, K.; Lyeth, B. G.; Gurkoff, G. G.; Muizelaar, J. P. and Berman, R. F. Neuroprotective Effects of Selective N-Type VGCC Blockade on Stretch-Injury-Induced Calcium Dynamics in Cortical Neurons. J. Neurotrauma. 2010, January, 27(1), 175-187.
Todd, P. A. and Fitton, A. Gallium nitrate. A review of its pharmacological properties and therapeutic potential in cancer related hypercalcemia. Drugs. 1991, August, 42(2), 261-73.
Warren, R. P.; Brockman, R. S.; Coonley, C. J.; Isaacs, M. and Staszewski, H. Gallium nitrate inhibits calcium resorption from bone and is effective treatment for cancer-related hypercalcemia. J. Clinical Investigation. 1984, 73, 1487-1490.

I claim:

1. A method of treating concussion or other brain trauma in humans or other mammals, comprising administering to the human or other mammal to be treated, an effective dose of a gallium compound or gallium complex.

2. The method of claim 1 wherein the gallium compound or complex is selected from the group consisting of: gallium nitrate, gallium citrate, gallium chloride, gallium fluoride, gallium phosphate, gallium carbonate, gallium acetate, gallium tartrate, gallium oxalate, gallium formate, gallium oxide, gallium sulfate, gallium arsenide, gallium maltolate, gallium 8-quinolinolate and hydrated gallium oxide, gallium pyridinones, gallium succinate, gallium gluconate, gallium 3-hydroxy-4-pyrone, gallium palmitrate, the tridocosahexaenonic acid salt of gallium, or other tri(alkylcarboxylato) gallium (III) compounds, gallium prophyrins, gallium transferrins, gallium pyridoxal isonicotinoyl hydrazine, or combinations thereof.

3. The method of claim 1 wherein said gallium compound or complex is administered orally, by spray, intravenously, subcutaneously, transdermally, intramuscularly, via bioadhesive patch, via mucoadhesive patch, via a buccal patch, or through oral or nasal inhalation.

4. The method of claim 1 wherein said gallium compound or complex is administered after the onset of said concussion or brain trauma.

5. The method of claim 1 wherein the gallium compound is administered in an amount sufficient to maintain steady state blood concentrations.

6. The method of claim 1 wherein the amount of gallium compound or complex administered is about 0.5 to about 20.0 mg/kg/day of body weight.

7. The method of claim 1 wherein said gallium compound is gallium nitrate.

8. The method of claim 7 wherein said gallium compound is administered orally, by spray, intravenously, subcutaneously, transdermally, intramuscularly, via bioadhesive patch, via mucoadhesive patch, via a buccal patch or through oral or nasal inhalation.

9. The method of claim 7 wherein said gallium nitrate is administered after the onset of said concussion or brain trauma.

10. The method of claim 7 wherein the gallium compound is administered in an amount sufficient to maintain steady state blood concentrations.

11. The method of claim 7 wherein the amount of gallium administered is about 0.5 to about 20.0 mg/kg/day of body weight with the preferred amount being 200 mg/m²/day.

12. The method of claim 1 wherein said gallium compound is a docosahexaenonic acid salt of gallium with one to three of the gallium ligands being from docosahexaenonic acid.

13. The method of claim 12 wherein said gallium compound is administered orally, by spray, intravenously, subcutaneously, transdermally, intramuscularly, via bioadhesive patch, via mucoadhesive patch, via a buccal patch or through oral or nasal inhalation.

14. The method of claim 12 wherein said docosahexaenonic acid salt of gallium is administered after the onset of said concussion or brain trauma.

15. The method of claim 12 wherein the gallium compound is administered in an amount sufficient to maintain steady state blood concentrations.

16. The method of claim 12 wherein the amount of gallium administered is about 0.5 to about 20.0 mg/kg/day of body weight.

17. The method of claim 1 wherein a human is being treated.

18. The method of claim 1 wherein a mammal other than a human is being treated.

19. The method of claim 1 wherein the gallium compound or complex is contained in a transdermal patch that may be applied to the skin after onset of the concussion or other brain trauma to permit rapid delivery of the compound through the skin.

20. The method of claim 1 wherein the gallium compound or complex is supplied from first aid kits for rapid treatment.
